(19) United States
(12) Patent Application Publication
(10) Pub. No.: US 2002/0183656 A1
(43) Pub. Date: Dec. 5, 2002

(54) Optimal dynamic techniques for custom-fit hip replacements

(75) Inventors: Menachem Levanoni, Yorktown Heights, NY (US); Jerome M. Kurtzberg, Yorktown Heights, NY (US)

Correspondence Address: IBM CORPORATION, INTELLECTUAL PROPERTY LAW DEPT., P.O. BOX 218, YORKTOWN HEIGHTS, NY, 10598, US

(73) Assignee: International Business Machines Corporation, Armonk, NY (US)

(21) Appl. No.: 09/874,411

(22) Filed: Jun. 5, 2001

(51) Int. Cl. A61B 5/103; A61B 5/117

(52) U.S. Cl. 600/587

(57) ABSTRACT

A computer method for providing optimal dynamic techniques for custom-fit hip replacements. The method includes steps of mounting pressure and acceleration sensors in a hip-enclosing device, transmitting data produced by said sensors during actual operation of said hip-enclosing device worn by a specific individual, receiving said sensor signals for subsequent analysis by a computer, creating a stress-and-acceleration map based on said sensor-based data, and creating a virtual orthodic (model) for optimal support and comfort based on step iv stress-and-acceleration map.

### BACKGROUND OF THE INVENTION

1. Field of the Invention

This invention relates to methodology for utilizing continual sensor-based data to design and adjust orthodics to fit an individual, in a given dynamic environment, in an optimal manner.

2. Introduction to the Invention

Static fitting techniques to design and construct orthodics for specific people are known. A plaster cast is taken and the orthodic is produced based on that plastic impression. However, no attention is given to the dynamic workings of the hip in the changing real environment. Specifically, the stresses and accelerations experienced by the hip during normal operation are not taken into account, nor is the optimum balance, between support and comfort, taken into account.

### SUMMARY OF THE INVENTION

We have now discovered novel methodology for exploiting the advantages inherent generally in sensing the dynamic workings (stresses) on specific hips/hips in actual motion, and using the sensor-based data to optimize the design and construction of the desired orthodics.

Our work proceeds in the following way.

We have recognized that a typical and important paradigm for presently effecting orthodics construction, is a largely static and subjective, human paradigm, and therefore exposed to all the vagaries and deficiencies otherwise attendant on static and human procedures. Instead, the novel paradigm we have in mind works in the following way:

First, a patient wears a set of pressure and acceleration sensors mounted, say, inside a hip-encasing device. These sensors record their associated stesses and accelerations produced in normal individual motion in its dynamic environment for a prescribed period of time sufficient to capture all possible stress and accelaration patterns.

The dynamically acquired data are fed into a computer which creates a map of the forces and accelerations experienced by the examined hip. This information is used to design an optimal orthodic which maximizes support and minimizes discomfort, and results in a computer production of a virtual orthodics that offers optimal performance to the examined hip in its normal operation.

A physical orthodic is then produced from a model provided by the virtual orthodic. This physical orthodic provides maximum support and maximal comfort to its wearer, following the optimal design of the orthodic.

We now itemize a novel computer method which can preserve the advantages inherent in the static approach, while minimizing the incompleteness and attendant static nature and subjectivities that otherwise inure in a technique heretofore used.

To this end, in a first aspect of the present invention, we disclose a novel computer method comprising the steps of:

i) mounting pressure and acceleration sensors in a hip-enclosing device;

ii) transmitting data produced by said sensors during actual operation of said hip-enclosing device worn by a specific individual;

iii) receiving said sensor signals for subsequent analysis by a computer;

iv) creating a stress-and-acceleration map based on said sensor-based data; and

v) creating a virtual orthodic (model) for optimal support and comfort based on step iv stress-and-acceleration map.

The novel method preferably comprises a further step of actually constructing said physical orthodic.

### BRIEF DESCRIPTION OF THE DRAWING

The invention is illustrated in the accompanying drawing, in which

FIG. 1 (numerals 10-34) provides an illustrative flowchart comprehending overall realization of the method of the present invention, including details of individual components.

### DETAILED DESCRIPTION OF THE PRESENT INVENTION

### Typical Application

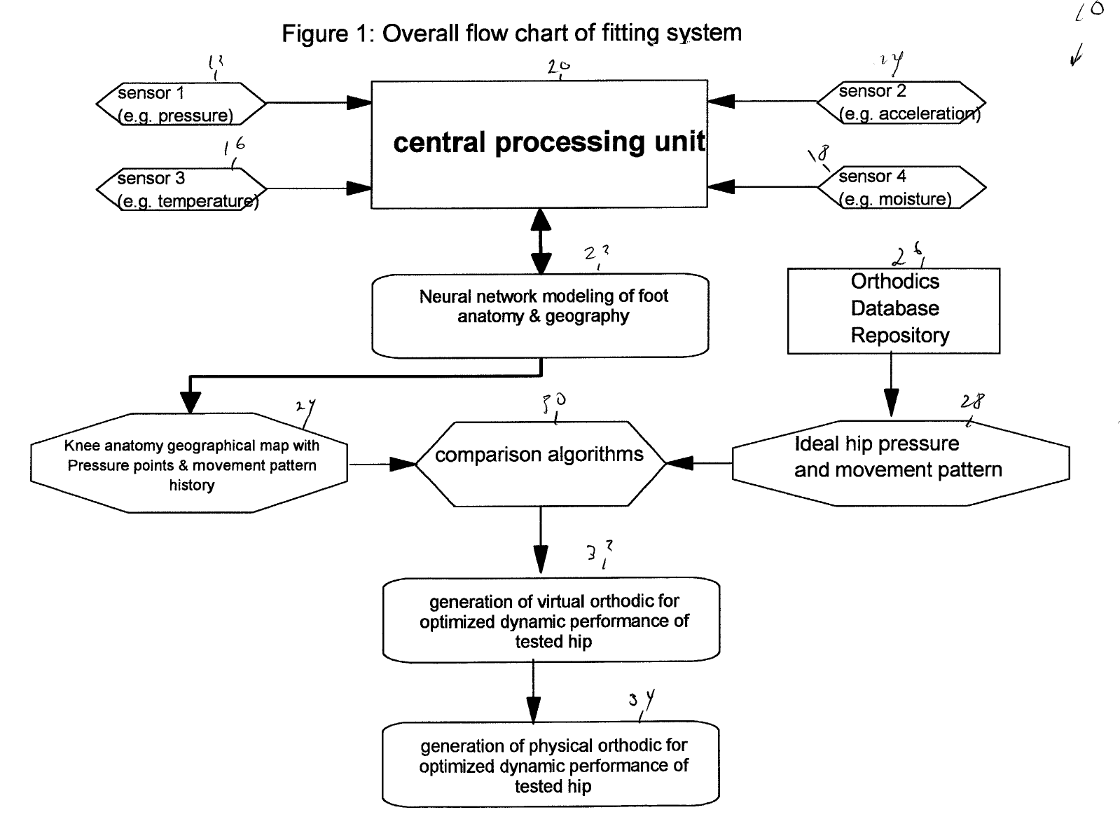

In a typical case (and with reference to FIG. 1), the patient's hip is fitted with a temporary device containing a number of sensors, located at prescribed locations on the tested hip. These sensors, which include pressure, acceleration, temperature, and humidity, are connected to a recording device.

The patient is asked to wear the device for several days and follow his/her normal routine.

During the test period, sensors data are recorded (including time stamps) in the recording device. The patient returns the device and the recording device at the end of the test period. The information stored in the recording device is then downloaded to a computer which stores all data in a database.

The data are then analyzed by a program (prefearably a neural network modeling program) which creates maps of the tested hip at different times. These maps also contains the sensors' reading at these times. Thus the system now has information on the dynamic behavior of the tested hip, including parametric information.

Based on these maps and maps of an ideal hip under similar conditions, an optimization program designs an optimized virtual orthodic for the patient. This design is then fed to a machine which generates an optimized physical orthodic.

What is claimed:

1. A computer method comprising the steps of:
i) mounting pressure and acceleration sensors in a hip-enclosing device;
ii) transmitting data produced by said sensors during actual operation of said hip-enclosing device worn by a specific individual;
iii) receiving said sensor signals for subsequent analysis by a computer;
iv) creating a stress-and-acceleration map based on said sensor-based data; and
v) creating a virtual orthodic (model) for optimal support and comfort based on step iv stress-and-acceleration map.

2. A method according to claim 1, comprising a step of using a temperature sensor which may be correlated with support and comfort of a worn orthodic.

3. A method according to claim 1, comprising a step of using an interpolation technique to completely map stresses and accelerations experienced by a hip over a period of time.

4. A method according to claim 3, comprising a step of updating the virtual orthodic model using the interpolating map.

5. A method according to claim 3, comprising a step of using the interpolated map to directly design the virtual orthodic in an optimal manner.

6. A method according to claim 1, comprising a step of a non-linear technique to model an orthodic.

7. A method according to claim 6, comprising a step of employing neural networks as the modeling technique.

8. A method according to claim 6, comprising a step of employing regression as the modeling technique.

9. A program storage device readable by machine, tangibly embodying a program of instructions executable by the machine to perform method steps for providing a custom hip replacement, the method comprising the steps of:
i) mounting pressure and acceleration sensors in a hip-enclosing device;
ii) transmitting data produced by said sensors during actual operation of said hip-enclosing device worn by a specific individual;
iii) receiving said sensor signals for subsequent analysis by a computer;
iv) creating a stress-and-acceleration map based on said sensor-based data; and
v) creating a virtual orthodic (model) for optimal support and comfort based on step iv stress-and-acceleration map.
